(19) United States
(12) Patent Application Publication
(10) Pub. No.: US 2025/0271522 A1
(43) Pub. Date: Aug. 28, 2025

(54) METASURFACE DESIGN APPARATUS AND METHOD

(71) Applicant: Seoul National University R&DB Foundation, Seoul (KR)

(72) Inventors: Namkyoo Park, Seoul (KR); Sunkyu Yu, Seoul (KR); Gyoungsub Yoon, Seoul (KR)

(73) Assignee: Seoul National University R&DB Foundation, Seoul (KR)

(21) Appl. No.: 19/066,005

(22) Filed: Feb. 27, 2025

(30) Foreign Application Priority Data
Feb. 28, 2024 (KR) 10-2024-0028542
Jan. 13, 2025 (KR) 10-2025-0005055

(51) Int. Cl. G01R 33/54; A61B 5/00; G01R 33/48

(52) U.S. Cl. CPC G01R 33/543; A61B 5/0042; G01R 33/4806

(57) ABSTRACT

A metasurface design apparatus and method are provided. A metasurface design apparatus according to an embodiment of the present invention is a metasurface design apparatus for designing a metasurface that is applied to a seven-tesla (7T) brain magnetic resonance imaging method, the metasurface design apparatus including a memory in which at least one instruction is stored, and a processor configured to execute the at least one instruction stored in the memory, wherein the processor acquires design information about an artificial magnetic field scatterer, acquires a plurality of high-frequency magnetic field (B₁⁺) modes distinguished from each other using the acquired design information, detects an optimal combination of the high-frequency magnetic field modes for homogenizing a high-frequency magnetic field, and designs an optimal metasurface based on the detected optimal combination.

### CROSS-REFERENCE TO RELATED APPLICATION

This application claims priority to and the benefit of Korean Patent Application No. 10-2024-0028542, filed on Feb. 28, 2024, and Korean Patent Application No. 10-2025-0005055, filed on Jan. 13, 2025 the disclosures of which are incorporated herein by reference in its entirety.

### BACKGROUND

### 1. Field of the Invention

The present invention relates to a metasurface design apparatus and method, and more particularly, to a metasurface design apparatus and method for designing a metasurface that is applied to a seven-tesla (7T) brain magnetic resonance imaging method.

### 2. Discussion of Related Art

Magnetic resonance imaging (MRI) is a medical imaging technology for noninvasively visualizing the internal structure of the human body. In MRI, when a uniform main static magnetic field B₀ is applied, with respect to a collective magnetic moment formed when nuclear spins in the human body are aligned along B₀, a B₁⁺ pulse with a frequency matching the Larmor frequency of a specific nuclide (composed mainly of hydrogen) is additionally applied from a radio frequency (RF) coil to induce resonance, and a magnetic resonance signal generated due to the resonance is converted into digital information and imaged.

In particular, ultrahigh field (UHF) MRI is a technology using a strong main static magnetic field (|B₀|≥7T), and a signal-to-noise ratio (SNR) and resolution of an image are considerably improved due to a magnetic resonance signal that increases according to the intensity of B₀. However, in UHF MRI, according to the Larmor frequency increasing in proportion to the intensity of B₀, higher frequency B₁⁺ pulses should be applied. Thus, since the approximate wavelength of electromagnetic waves transmitted into the human tissue is similar to or smaller than the size of the skull, inhomogeneity of B₁⁺ occurs and causes inhomogeneity of a received magnetic resonance signal, resulting in a degradation in image quality. Similarly, the inhomogeneity of a generated high-frequency electric field causes an increase in local electric field intensity, which increases a specific absorption rate (SAR) in the human tissue.

The background of the present invention is disclosed in Korean Patent Publication No. 10-1953350 (published on Feb. 22, 2019).

### SUMMARY OF THE INVENTION

The present invention is directed to providing a metasurface design apparatus and method with which an optimal metasurface for achieving wide area homogenization of a high-frequency magnetic field transmitted to a brain of a subject in a seven-tesla (7T) brain magnetic resonance imaging (MRI) method can be designed.

The present invention is also directed to providing a metasurface design apparatus and method in which a portion of a metasurface of which the importance is low is removed through pruning to simplify a structure of the metasurface, and stability is imparted to B₁⁺ uniformity to allow the metasurface to be highly compatible with various subjects.

However, the technical objects to be solved by the present invention are not limited to the above, and other objects that are not described herein will be clearly understood by those skilled in the art from the following descriptions of the present invention.

According to an aspect of the present invention, there is provided a metasurface design apparatus for designing a metasurface that is applied to a 7T brain MRI method, the metasurface design apparatus including a memory in which at least one instruction is stored, and a processor configured to execute the at least one instruction stored in the memory, wherein the processor acquires design information about an artificial magnetic field scatterer, acquires a plurality of high-frequency magnetic field (B₁⁺) modes distinguished from each other using the acquired design information, detects an optimal combination of the high-frequency magnetic field modes for homogenizing a high-frequency magnetic field, and designs an optimal metasurface based on the detected optimal combination.

The artificial magnetic field scatterer may have an elliptical cylindrical structure including copper wires and parallel plate capacitors which are regularly arranged.

Effective material properties of the artificial magnetic field scatterer and magnetic field scattering by the artificial magnetic field scatterer may be determined by a capacitance value of the parallel plate capacitor.

In the high-frequency magnetic field mode, a distribution of a high-frequency magnetic field induced inside a subject may be exhibited by a radio frequency (RF) coil and the artificial magnetic field scatterer.

A region of interest for the homogenizing may be an entire brain region.

The processor may acquire the plurality of high-frequency magnetic field modes by repeatedly performing a process of modeling the artificial magnetic field scatterer based on the design information about the artificial magnetic field scatterer to generate an artificial magnetic field scatterer model, arranging the generated artificial magnetic field scatterer model in a virtual three-dimensional space, and then acquiring information about a distribution of a high-frequency magnetic field induced inside a subject while moving the artificial magnetic field scatterer model a preset distance in a preset direction.

The homogenizing of the high-frequency magnetic field may be defined by a linear combination of complex-valued weights for each of the plurality of high-frequency magnetic field modes.

The complex-valued weights may be determined through a cost function for minimizing a coefficient of variation for a distribution of an absolute value of the high-frequency magnetic field in a region of interest for the homogenizing, and the processor may adjust the complex-valued weights in a direction in which the coefficient of variation is minimized using a gradient descent method.

The processor may repeatedly perform a process of deriving a complex-valued weight that minimizes the coefficient of variation, calculating importance of each of the plurality of high-frequency magnetic field modes, identifying a high-frequency magnetic field mode of which the importance is lowest, and removing the identified high-frequency magnetic field mode until the high-frequency magnetic field mode is no longer present, and then the processor may identify a combination of high-frequency magnetic field modes of which the coefficient of variation is smallest and may detect the identified combination as the optimal combination.

The importance may be determined according to an absolute value of the complex-valued weight.

When there are a plurality of combinations of high-frequency magnetic field modes of which the coefficient of variation is smallest, a combination in which a number of included high-frequency magnetic field modes is smallest may be detected as the optimal combination.

The processor may determine a metasurface with a structure of a combination of magnetic field scatterers that induce the optimal combination of the high-frequency magnetic field modes as the optimal metasurface.

According to another aspect of the present invention, there is provided a metasurface design method of designing a metasurface that is applied to a 7T brain MRI method, the metasurface design method including acquiring, by a processor, design information about an artificial magnetic field scatterer, acquiring, by the processor, a plurality of high-frequency magnetic field (B₁⁺) modes distinguished from each other using the artificial magnetic field scatterer, detecting, by the processor, an optimal combination of the high-frequency magnetic field modes for homogenizing a high-frequency magnetic field, and designing, by the processor, an optimal metasurface based on the detected optimal combination.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which: FIG. 1 is a block diagram illustrating a metasurface design apparatus according to an embodiment of the present invention;

FIGS. 2A to 2F show exemplary diagrams for describing a structure and an operating principle of an artificial magnetic field scatterer according to an embodiment of the present invention;

FIGS. 3A to 3C show exemplary diagrams for describing a method of transforming an artificial magnetic field scatterer according to an embodiment of the present invention;

FIGS. 4A to 4C show exemplary diagrams for describing a process of designing an artificial magnetic field scatterer applicable to a seven-tesla (7T) magnetic resonance imaging method;

FIGS. 5A to 5C show exemplary diagrams for describing a process of deriving an optimal metasurface through pruning;

FIG. 6 is an exemplary diagram for describing a process of actually implementing an optimal metasurface;

FIG. 7 is an exemplary diagram for describing a structure of a metasurface reflecting a combination of optimized effective material properties;

FIG. 8 shows exemplary diagrams for describing a distribution of a high-frequency magnetic field induced inside a subject when an optimized metasurface is applied;

FIGS. 9A and 9B show exemplary diagrams for describing that a metasurface optimized for a specific head has excellent compatibility with various subjects;

FIG. 10 is a first flowchart illustrating a metasurface design method according to an embodiment of the present invention; and

FIG. 11 is a second flowchart illustrating a metasurface design method according to an embodiment of the present invention.

### DETAILED DESCRIPTION OF EXEMPLARY EMBODIMENTS

Hereinafter, embodiments of the present invention will be described with reference to the accompanying drawings. Prior to the description, it should be understood that the terms used in the specification and the appended claims should not be construed as limited to general and dictionary meanings, but interpreted based on the meanings and concepts corresponding to technical aspects of the present invention on the basis of the principle that the inventor is allowed to define terms appropriately for the best description. Accordingly, embodiments disclosed in the present specification and configurations illustrated in the drawings are merely most exemplary embodiments of the present invention and do not represent all of the technical ideas of the present invention, and thus it should be understood that there may be various equivalents and modifications that may substitute these at the time of filing of the present application. Further, “comprise,” include,” “comprising,” and/or “including” used in this specification should be interpreted as specifying the presence of described shapes, numbers, steps, operations, members, elements, and/or groups thereof and do not exclude the presence or addition of other shapes, numbers, operations, members, elements, and/or groups thereof. Further, the use of “may” and “may be” when describing embodiments of the present invention refers to “one or more embodiments of the present invention.”

In addition, for a better understanding of the present invention, the accompanying drawings are not illustrated on an actual scale and sizes of some elements may be exaggerated. In some embodiments, the same reference numbers may be assigned to the same components in different embodiments.

Stating that two objects of comparison are “the same” means that the two objects of comparison are “substantially the same.” Therefore, substantially the same may include a deviation that is considered low in the art, for example, a deviation of 5% or less. In addition, uniformity of a parameter in a certain area may mean uniformity from an average perspective.

It will be understood that, although the terms first, second, and the like are used herein to describe various components, these components should not be limited by these terms. These terms are only used to distinguish one component from another component, and a first component may also be a second component unless particularly described otherwise.

Through the specification, each component may be singular or plural unless particularly described otherwise.

When it is said that an arbitrary element is disposed on “an upper portion (or a lower portion)” of an element or disposed “above (or below)” an element, this may not only mean that the arbitrary element is disposed in contact with an upper surface (or a lower surface) of the element, but also that another element may be interposed between the element and the arbitrary element disposed above (or below) the element.

Also, when it is said that a certain element is “connected” or “coupled” to another component, this may mean that the components are directly connected or coupled to each other, but it should be understood that another component may be “interposed” between the components or the components may be “connected” or “coupled” to each other via another component. Further, the term “electrically coupled” may mean not only “directly coupled” but may also include “coupled via another interposing component.”

Whenever reference is made throughout the specification to “A and/or B,” this means A, B, or A and B, unless otherwise specified. That is, “and/or” includes all or any combination of a plurality of listed items. “C to D” refers to C or more and D or less unless particularly described otherwise.

FIG. 1 is a block diagram illustrating a metasurface design apparatus according to an embodiment of the present invention.

Referring to FIG. 1, a metasurface design apparatus 100 according to an embodiment of the present invention may include a communication interface 110, a user interface 120, a memory 130, and a processor 140. The metasurface design apparatus 100 according to the embodiment of the present invention may further include various components in addition to the components shown in FIG. 1.

The communication interface 110 may communicate with an external device. The communication interface 110 may communicate with various types of external devices according to various types of communication methods. Various types of information required in a process of designing a metasurface may be received through the communication interface 110.

The user interface 120 may be a device for interacting with a user. The user interface 120 may include an input device for receiving a user input. The input device may include a keyboard, a mouse, a touch screen, or the like. The user interface 120 may include an output device for outputting information. The output device may include a monitor, a speaker, a light-emitting diode (LED), or the like. Various types of information required in a process of designing a metasurface may be received through the user interface 120.

The memory 130 may store at least one command that is executed in a process in which the processor 140 to be described below designs a metasurface. The memory 130 may store basic data required for designing a metasurface or data generated during a process in which the processor 140 designs a metasurface, and the processor 140 may access data stored in the memory 130 to perform a metasurface design operation. The memory 130 may be implemented as a computer-readable recording medium and may operate such that the processor 140 may access the memory 130. Specifically, the memory 130 may be implemented as an optical data storage device such as a hard drive, a magnetic tape, a memory card, a read-only memory (ROM), a random-access memory (RAM), a digital video disc (DVD), or an optical disc.

The processor 140 may be a device that performs an operation of designing a metasurface, may be implemented as an application specific integrated circuit (ASIC), a digital signal processor (DSP), a programmable logic device (PLD), a field programmable gate array (FPGS), a central processing unit (CPU), a microcontroller, and/or a microprocessor, and may drive an operating system or application and control a plurality of hardware or software components. The processor 140 may be configured to execute at least one instruction stored in the memory 130 and store execution result data in the memory 130.

The processor 140 may acquire design information about an artificial magnetic field scatterer, may acquire a plurality of high-frequency magnetic field (B₁⁺) modes distinguished from each other using the acquired design information, may detect an optimal combination of the high-frequency magnetic field modes for homogenizing a high-frequency magnetic field, and may design an optimal metasurface based on the detected optimal combination.

The artificial magnetic field scatterer may include copper wires and parallel plate capacitors which are regularly arranged. A structure of the artificial magnetic field scatterer may be an elliptical cylinder structure. The effective material properties of the artificial magnetic field scatterer and the magnetic field scattering by the artificial magnetic field scatterer may be determined by a capacitance value of the parallel plate capacitor.

In the high-frequency magnetic field mode, a distribution of a high-frequency magnetic field induced inside a subject (for example, a human head) by a radio frequency (RF) coil and the artificial magnetic field scatterer may be exhibited. A region of interest (ROI) for homogenizing may be the entire brain region.

While moving an artificial magnetic field scatterer model a preset distance (for example, 3 cm) in a preset direction (for example, a vertical direction), the processor 140 may repeatedly perform a process of modeling an artificial magnetic field scatterer based on design information about the artificial magnetic field scatterer to generate the artificial magnetic field scatterer model, arranging the generated artificial magnetic field scatterer model in a virtual three-dimensional space, and then acquiring information about a distribution of a high-frequency magnetic field induced inside a subject, thereby acquiring a plurality of high-frequency magnetic field modes.

The homogenizing of a high-frequency magnetic field may be defined by a linear combination of complex-valued weights for each of a plurality of high-frequency magnetic field modes. In this case, the complex-valued weights for each of the plurality of high-frequency magnetic field modes may be determined through a cost function for minimizing a coefficient of variation (CV) for a distribution of an absolute value |B₁⁺| of the high-frequency magnetic field in an ROI for homogenizing. By using a gradient descent method, the processor 140 may adjust the complex-valued weight in a direction in which the CV is minimized.

The processor 140 may repeatedly perform a process of deriving a complex-valued weight that minimizes a CV, calculating the importance of each of a plurality of high-frequency magnetic field modes, identifying a high-frequency magnetic field mode of which the calculated importance is the lowest, and removing the identified high-frequency magnetic field mode until a high-frequency magnetic field mode is no longer present. That is, the processor 140 may check the CV while changing a combination of high-frequency magnetic field modes (removing a high-frequency magnetic field mode, of which the importance is the lowest, from the combination one by one). The importance of the high-frequency magnetic field mode may be determined according to an absolute value |Cₙ| of a complex-valued weight. The processor 140 may identify a combination of high-frequency magnetic field modes of which a CV is the smallest and may detect the identified combination as an optimal combination. When there are a plurality of combinations of high-frequency magnetic field modes of which a CV is the smallest, the processor 140 may detect a combination in which the number of included high-frequency magnetic field modes is the smallest as the optimal combination.

The processor 140 may determine a metasurface, which has a structure of a combination of magnetic field scatterers that induce an optimal combination of high-frequency magnetic field modes, to be an optimal metasurface (a metasurface with an optimal structure). That is, a metasurface designed according to the present embodiment may be a combination of magnetic field scatterers that induce an optimal combination of high-frequency magnetic field modes.

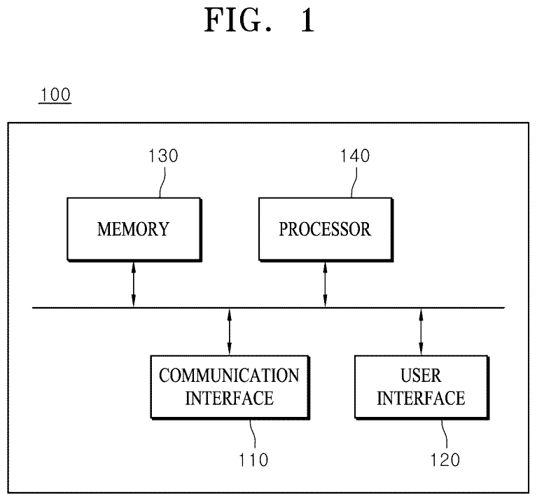

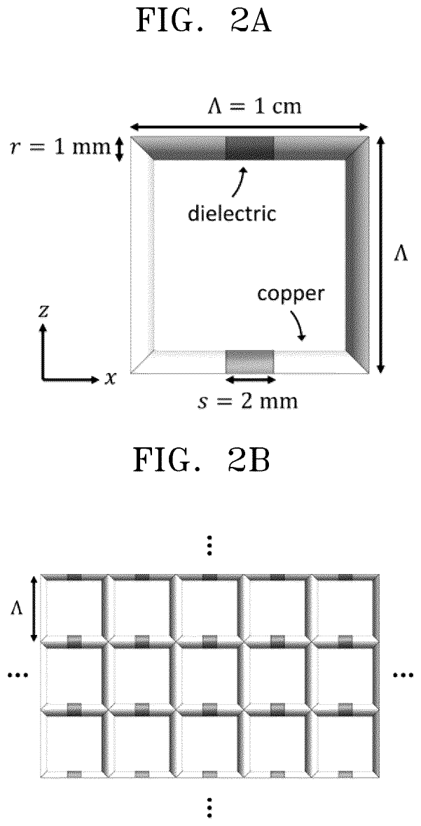

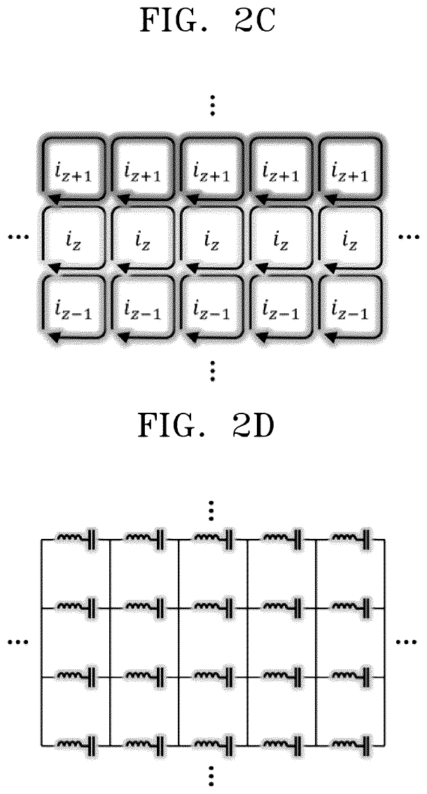

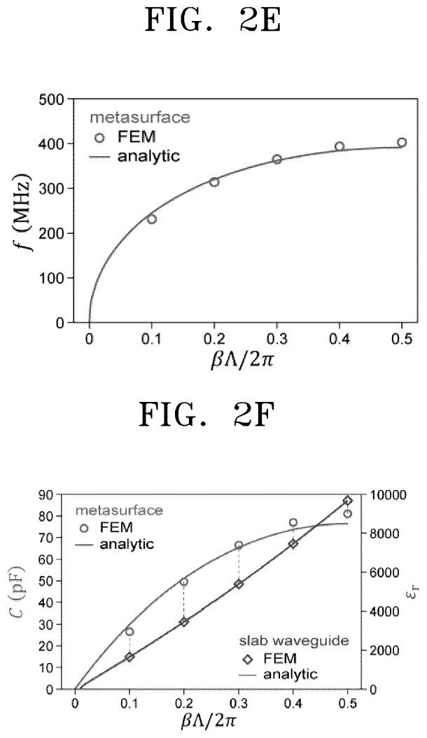

FIGS. 2A to 2F show exemplary diagrams for describing a structure and an operating principle of an artificial magnetic field scatterer according to an embodiment of the present invention.

The artificial magnetic field scatterer may include a plurality of unit cells. As shown in FIG. 2A, the unit cell may have a quadrangular structure in which two copper wires having a “⊂” shape with a radius r of 1 mm are connected in a loop shape. A dielectric having a length s of 2 mm may be interposed between the two copper wires. A unit length ∧ of the unit cell may be 1 cm. However, the structure of the unit cell is not limited to the above-described embodiment, and various types of structures may be adopted as the structure of the unit cell.

As shown in FIG. 2B, the artificial magnetic field scatterer may have a planar structure in which identical unit cells are repeatedly arranged per unit length, and in this structure, the dielectrics and the copper wires may form parallel plate capacitors and inductors that are repeatedly arranged.

In FIG. 2C, when electromagnetic waves propagate in a z direction along the artificial magnetic field scatterer, a mesh current I_{z} of which a phase varies according to a z position may be induced in each loop corresponding to the unit cell. In this case, Bloch's theorem is applied to establish a relationship between adjacent mesh currents as shown in Equation 1 below. In Equation 1, I_{z} may denote a mesh current, β may denote a wavenumber, and may denote a unit length.

$${I}_{z + n} = {I}_{z} {e}^{- jβ nΛ} \qquad [ Equation 1 ]$$

As shown in FIG. 2D, a structure of the artificial magnetic field scatterer may be analyzed by being substituted with an equivalent circuit in which capacitors and inductors are repeatedly arranged. In this case, Kirchhoff's voltage law is applied to one loop with the mesh current I_{z} to establish a relationship between adjacent mesh currents as shown in Equation 2 below. In Equation 2 below, ω may denote an operating angular frequency, C may denote a capacitance, and M may denote a mutual inductance.

$$\frac{2 {I}_{z}}{C} - \frac{{I}_{z + 1} + {I}_{z - 1}}{C} - {ω}^{2} \sum {nI}_{z + n} {M}_{z + n} Λ = 0 \qquad [ Equation 2 ]$$

In Equation 2 above, M_{z+n} may denote a mutual inductance per unit horizontal length between one loop having a mesh current I_{z} and a parallel-wire transmission line through which a current I_{z+n} flows.

Equations 1 and 2 above are combined to establish a dispersion relationship of the structure of the artificial magnetic field scatterer as shown in Expression 3 below.

$$ω = {CM}^{′ - \frac{1}{2}} sin ( \frac{βΛ}{2} ) , {M}^{′} = \sum {ne}^{- j β n Λ} {M}_{z + n} Λ \qquad [ Equation 3 ]$$

β calculated from the dispersion relationship of the artificial scatterer structure may be compared with β derived from an analysis result through a finite element method (FEM), thereby proving the validity of the distribution relationship.

FIG. 2E shows a relationship between β and an operating frequency f of the structure of the artificial magnetic field scatterer. In this case, a C value is fixed at 45 pF, and a relative permittivity value εᵣ of a parallel plate capacitor dielectric required for the C value may be 3,236.

FIG. 2F shows a relationship between β and C of the structure of the artificial magnetic field scatterer. In this case, f may be fixed at 300 MHz which is an operating frequency (that is, a Larmor frequency) of a seven-tesla (7T) magnetic resonance imaging (MRI) method. In addition, β of the structure of the artificial magnetic field scatterer may be compared with β of a dielectric slab waveguide structure with the same thickness of 2 mm, thereby determining the effective material properties or effective permittivity of the structure of the artificial magnetic field scatterer.

When an operating frequency is fixed as in the 7T MRI method, the effective material properties of the structure of the artificial magnetic field scatterer may be adjusted through the C value of the parallel plate capacitor, and the C value of the parallel plate capacitor may be determined with a relative permittivity εᵣ) value of the parallel plate capacitor dielectric. In addition, in a process of designing an artificial magnetic field scatterer, which will be described below, it is necessary to achieve a target C value by using a parallel plate capacitor dielectric having a realistic εᵣ value rather than εᵣ value (that is, 3,236).

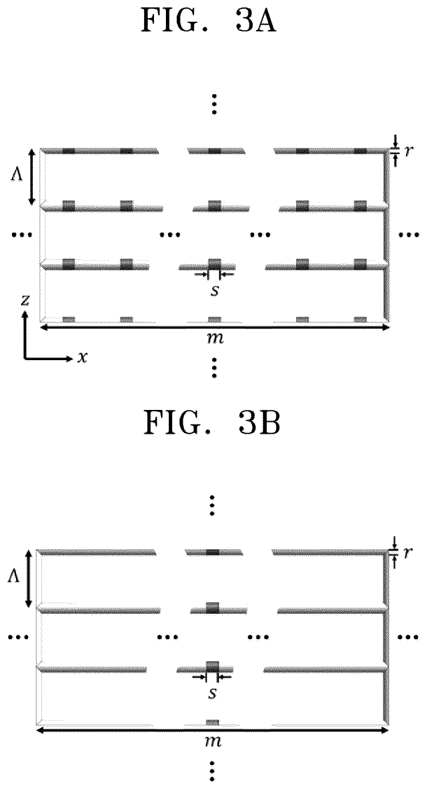

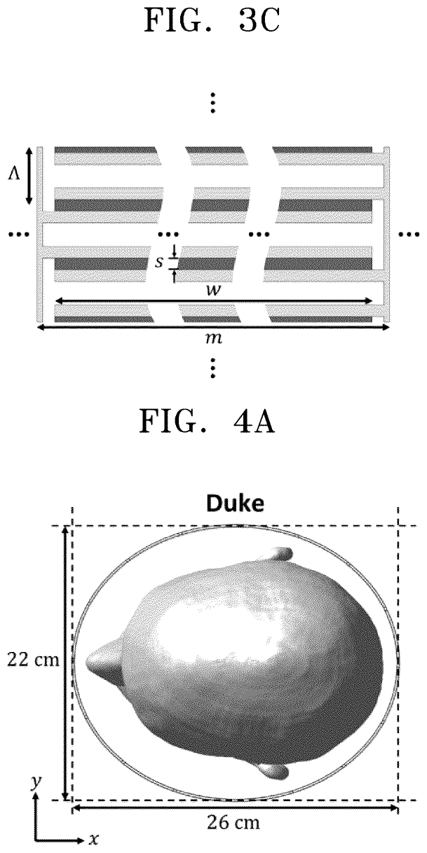

FIGS. 3A to 3C show exemplary diagrams for describing a method of transforming an artificial magnetic field scatterer according to an embodiment of the present invention.

As shown in FIG. 3A, when a phase of a mesh current induced in a structure of an artificial magnetic field scatterer is invariant in a horizontal direction (or x direction), currents flowing in a vertical direction of a wire may be canceled, thereby forming an open circuit between adjacent wires parallel to the horizontal direction.

As shown in FIG. 3B, when m parallel plate capacitors are connected in series along a wire while a length s is maintained, a value of equivalent capacitance C_{eq} may be reduced by a factor of m.

As shown in FIG. 3C, when an electrode surface area of a parallel plate capacitor is widened by replacing an existing cylindrical wire with a wire with a square cross section while maintaining s, a value of equivalent capacitance C_{eq} may be further reduced by a factor of n (=wt/πr²) according to a ratio between surface areas before and after transformation. Here, w may denote a width of an electrode, t may denote a thickness of a structure of an artificial magnetic field scatterer, and a thickness after transformation may be equal to a thickness of 2 mm before transformation.

Through the transformation of the structure of the artificial magnetic field scatterer, a C value required to achieve the target effective material properties may be lowered with a total reduction factor as much as m×n. In addition, since the C value and an εᵣ value of a parallel plate capacitor dielectric are proportional to each other, an εᵣ value required to achieve target effective material properties may also be lowered with the same reduction factor. That is, through the transformation of the structure of the artificial magnetic field scatterer, an artificial magnetic field scatterer may be designed using a parallel plate capacitor dielectric with a realistic εᵣ value.

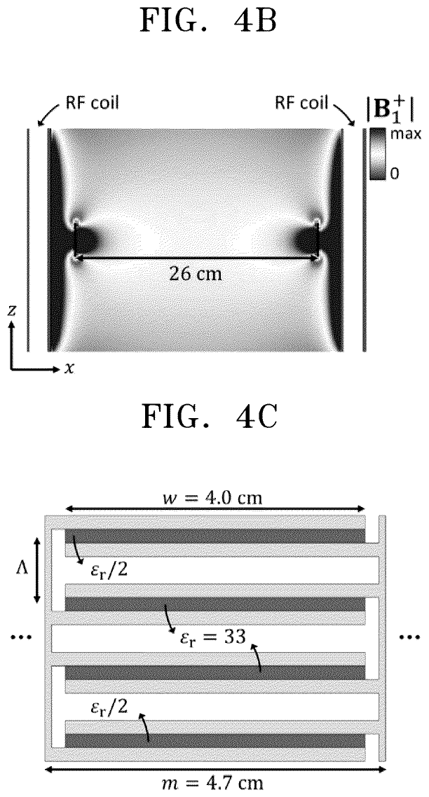

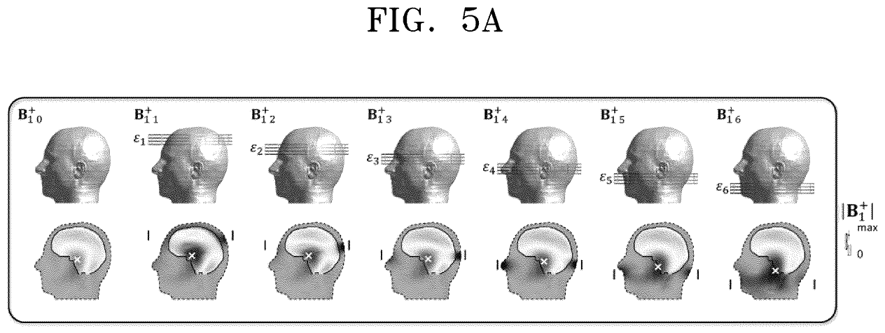

FIGS. 4A to 4C show exemplary diagrams for describing a process of designing an artificial magnetic field scatterer applicable to a 7T MRI method.

As shown in FIG. 4A, when applied to a 7T MRI method, a magnetic field scatterer may be disposed in a shape (elliptical shape) that surrounds a head. In this case, a major axis length and a minor axis length of the magnetic field scatterer may be set to values for effectively transmitting a scattered magnetic field to an elliptical head inside the magnetic field scatterer. For example, the major axis length of the magnetic field scatterer may be 26 cm, and the minor axis length thereof may be 22 cm. However, the major and minor axis lengths of the magnetic field scatterer are not limited to the above-described embodiments, and the major and minor axis lengths of the magnetic field scatterer may be set to various values according to the designer's intention.

As shown in FIG. 4B, a magnetic field formed by an external RF coil propagates inward and induces magnetic field scattering by the artificial magnetic field scatterer. In particular, when a structure of the artificial magnetic field scatterer satisfies half-wavelength resonance conditions, strong magnetic field scattering may be induced.

The RF coil used throughout the present embodiment may be a cylindrical 16-channel transverse electromagnetic (TEM) coil. A vertical length and an inner diameter of the RF coil may be set to values for providing sufficient space for the artificial magnetic field scatterer and a subject disposed inside the RF coil. For example, the vertical length of the RF coil may be 24 cm, and the inner diameter thereof may be 32 cm. However, the vertical length and internal diameter of the RF coil are not limited to the above-described embodiment, and the vertical length and internal diameter of the RF coil may be set to various values according to the designer's intention.

FIG. 4C illustrates an elliptical magnetic field scatterer functioning as a z-axis half-wave resonator. A structure of the elliptical magnetic field scatterer may be transformed to be divided into 16 equal parts in a horizontal direction in consideration of the number of channels of the RF coil (16 channels), and in this case, a reduction coefficient m may be 4.7. In addition, an electrode surface area of a parallel plate capacitor may be widened by maximizing a given space, thereby lowering an εᵣ value, which is required to satisfy half-wave resonance conditions, with a sufficiently large total reduction factor (that is, m×n). Considering that the structure including three unit cells with a unit length ∧ of 1 cm, a C value should be adjusted such that an effective half-wavelength in the structure becomes 3 cm, and according to a dispersion relationship of the artificial magnetic field scatterer, the C value may be 45 pF. When an εᵣ value required to satisfy a half-wave resonance condition is calculated with a reduction factor through the structural transformation of the elliptical magnetic field scatterer, and detailed adjustment is added to a value calculated in consideration of an error between a theoretically derived result and an actual result, a final εᵣ value may be 33.

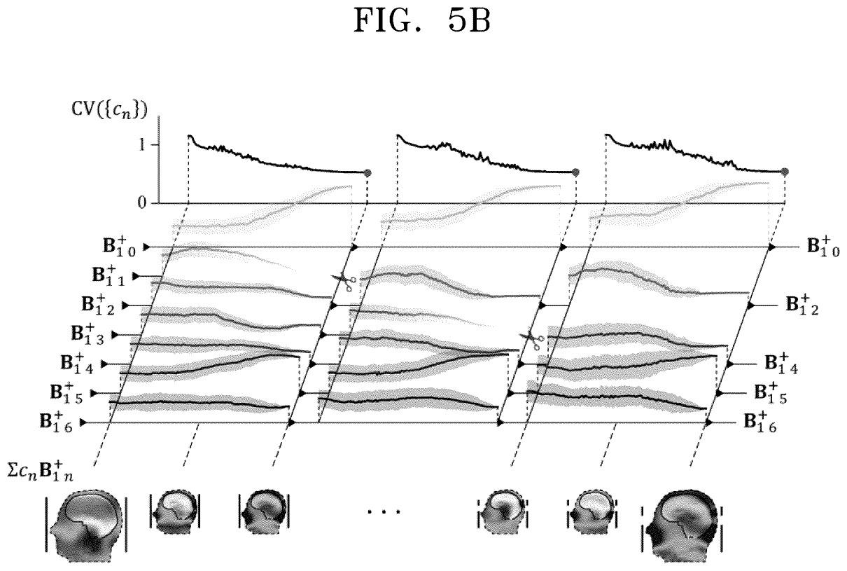

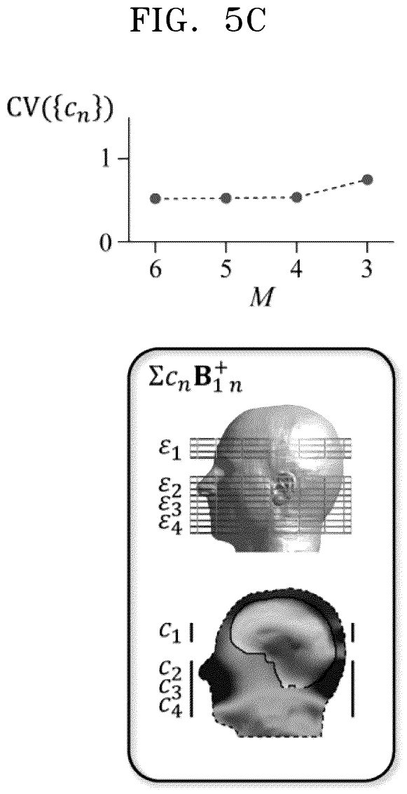

FIGS. 5A to 5C show exemplary diagrams for describing a process of deriving an optimal metasurface through pruning.

As shown in FIG. 5A, in a 7T MRI operating environment in which a virtual head model (Duke) is a subject, an elliptical artificial magnetic field scatterer used as a half-wave resonator may be disposed to surround a head, and a high-frequency magnetic field mode may be induced inside the subject by a magnetic field formed by an external RF coil and the artificial magnetic field scatterer. A processor 140 may acquire a plurality of high-frequency magnetic field modes that are distinguished from each other by checking a distribution of a high-frequency magnetic field induced inside the subject while moving the artificial magnetic field scatterer a preset distance (for example, 3 cm) in a preset direction (for example, a vertical direction). When an artificial magnetic field scatterer is not present, the processor 140 may also check a distribution B₁⁺₀ of a high-frequency magnetic field induced inside a subject (a high-frequency magnetic field induced inside the subject only by a magnetic field of an RF coil). An ROI, which is a target for which a high-frequency magnetic field is homogenized, may be the entire brain region, and a region indicated by a solid line in FIG. 5A may correspond to the ROI.

As shown in FIG. 5A, the homogenizing of the high-frequency magnetic field may be defined by a linear combination (ΣnCₙB₁⁺ₙ) of complex-valued weights Cₙ for each of a plurality of high-frequency magnetic field modes. Here, B₁⁺ₙ may denote a high-frequency magnetic field mode. Each complex-valued weight may be determined through a gradient descent method in which a cost function is set with a CV for a distribution of an absolute value of the high-frequency magnetic field |B₁⁺| inside the ROI. The importance of a magnetic field scatterer may be determined by a relative ratio with respect to an absolute value |Cₙ| of the complex-valued weight for each of the plurality of high-frequency magnetic field modes. The CV may be a representative index for evaluating the uniformity of a high-frequency magnetic field in MRI and may be a value calculated by dividing a standard deviation of a distribution of the absolute value |B₁⁺| of the high-frequency magnetic field within the ROI by an average. A small CV may mean that a high-frequency magnetic field is uniformly distributed, and a cost function to be minimized through a gradient descent method may be set with a CV, thus effectively achieving the homogenizing of a virtual high-frequency magnetic field.

In FIG. 5B, during a process in which a cost function converges, a dark line indicates an average of a relative ratio of |Cₙ| obtained by performing a gradient descent method, in which initial conditions (that is, Cₙ) are randomly set, 50 times, a width of a light color indicates an error range of |Cₙ|, and a corresponding normalized CV is shown. In FIG. 5B, B₁⁺ₙ of which the importance is the lowest (that is, the relative ratio of |Cₙ| is the lowest) is removed, and scissors indicate pruning.

FIG. 5C shows a CV according to the number of residual high-frequency modes B₁⁺ₙ (the number of high-frequency modes remaining after removal). As shown in FIG. 5C, a combination of high-frequency magnetic field modes of which a CV is the smallest may be detected as an optimal combination. When there are a plurality of combinations of high-frequency magnetic field modes of which a CV is the smallest, a combination in which the number of included high-frequency magnetic field modes is the smallest may be detected as the optimal combination. In the case of FIG. 5C, a combination when the number of high-frequency magnetic field modes is M may be detected as the optimal combination. When the number of high-frequency magnetic field modes is four, the number of magnetic field scatterers constituting a metasurface also becomes four.

In the present embodiment, a portion of a metasurface (high-frequency magnetic field mode) of which the importance is low may be removed through pruning to simplify a structure of the metasurface, and stability may be imparted to B₁⁺ uniformity to allow the metasurface to be highly compatible with various subjects.

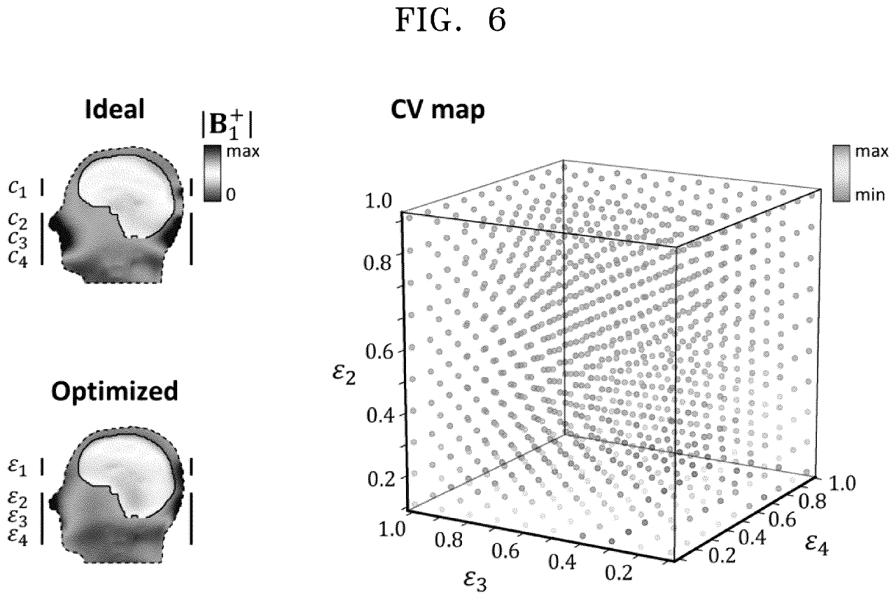

FIG. 6 is an exemplary diagram for describing a process of actually implementing an optimal metasurface.

Referring to FIG. 6, the processor 140 may apply a depth-first search method in a 7T MRI operating environment to adjust the effective material properties εₙ of each artificial magnetic field scatterer constituting a metasurface and the magnetic field scattering by the artificial magnetic field scatterers constituting the metasurface, thereby searching for actually implementable high-frequency magnetic field homogenization that approaches the most ideal high-frequency magnetic field homogenization. Specifically, the processor 140 may search for a CV of a |B₁⁺| distribution actually implemented according to a combination of effective material properties and may determine a case with the lowest CV as the optimal high-frequency magnetic field homogenization that approaches the most ideal high-frequency magnetic field homogenization. In this case, a combination of optimized effective material properties that induce optimal high-frequency magnetic field homogenization may determine a detailed structure of a metasurface. FIG. 6 illustrates the normalized effective material properties of each magnetic field scatterer constituting a metasurface, and ε₁ is omitted from the diagram.

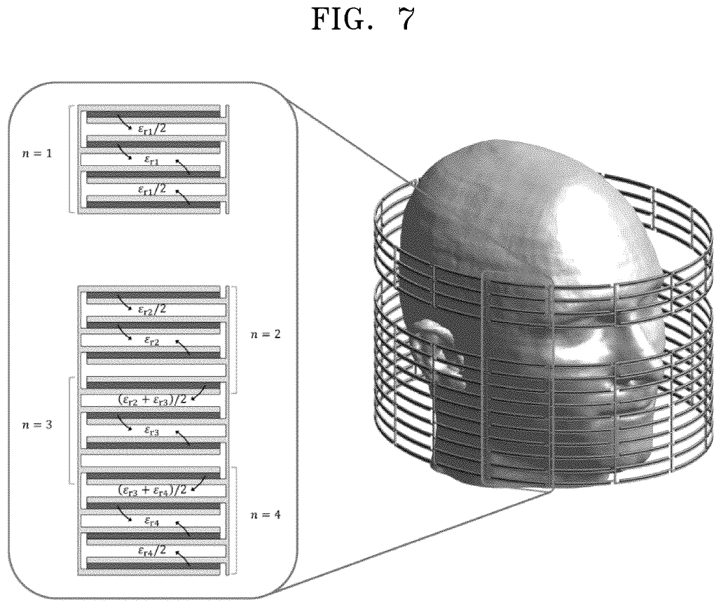

FIG. 7 is an exemplary diagram for describing a structure of a metasurface reflecting a combination of optimized effective material properties.

Referring to FIG. 7, the effective material properties of each magnetic field scatterer constituting the metasurface as described above are determined by the relative permittivity εᵣ of a capacitor dielectric, optimized εᵣ values are 23.1, 5.5, 22.0, and 12.1, and a major axis and a minor axis of the meta surface have lengths of 26 cm and 22 cm.

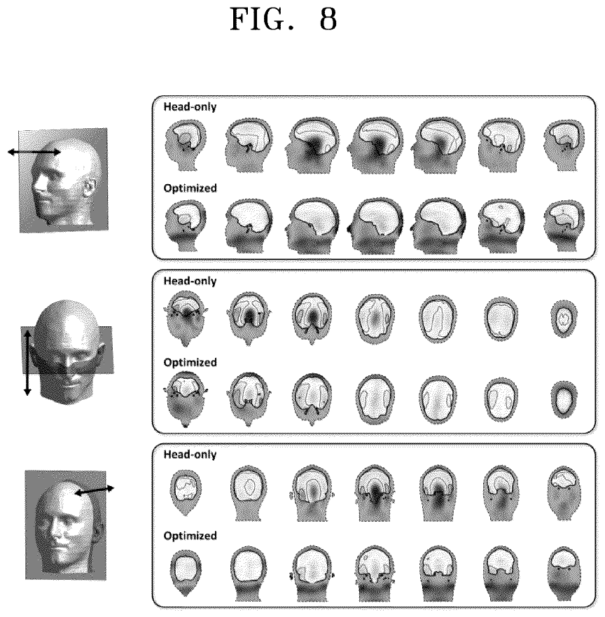

FIG. 8 shows exemplary diagrams for describing a distribution of a high-frequency magnetic field induced inside a subject when an optimized metasurface is applied.

Referring to FIG. 8, when a metasurface is not present (head-only case) and is present (optimized case) in a 7T MRI operating environment, a distribution of a high-frequency magnetic field is positioned in each of an upper section and a lower section, and a distribution of a high-frequency magnetic field is shown on a slice of a virtual head model (Duke) that varies along three axes. Here, solid lines each indicate a contour line in which a ratio of |B₁⁺| to the maximum value |B₁⁺|max of |B₁⁺| inside an ROI (that is, B₁⁺|/|B₁^{+|}ₘₐₓ) is ½ and ⅓, respectively.

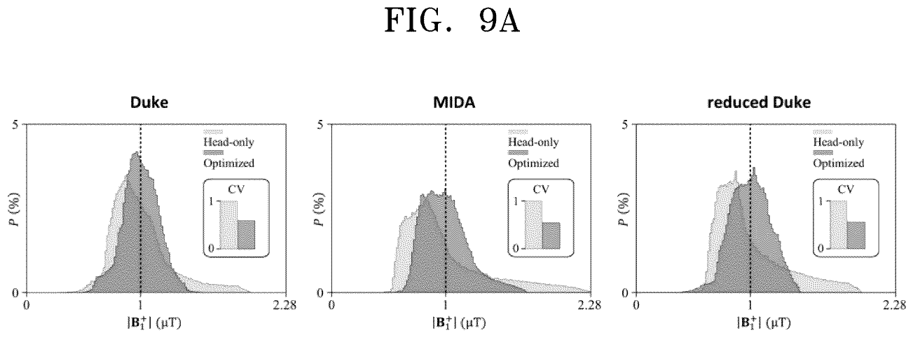

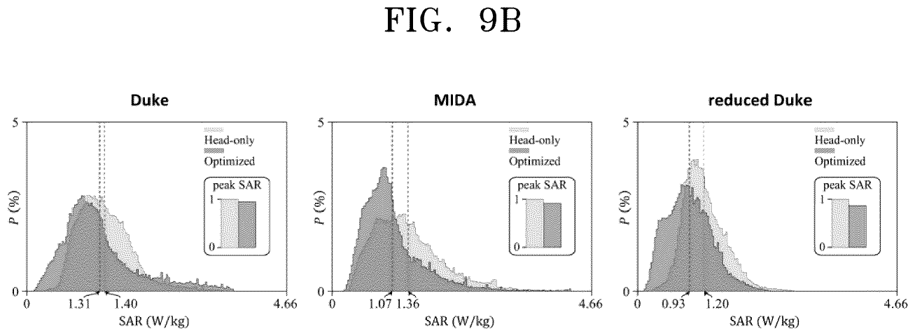

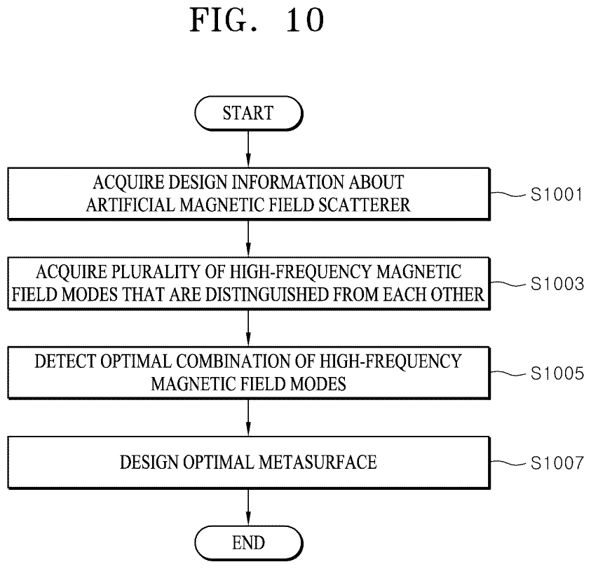

FIGS. 9A and 9B show exemplary diagrams for describing that a metasurface optimized for a specific head has excellent compatibility with various subjects.

As shown in FIG. 9A, when a metasurface optimized for Duke is not present (head-only case) and is present (optimized case) in a 7T MRI operating environment, a probability density function of a |B₁⁺| distribution inside an ROI of each of Duke (male head model), MIDA (female Mori model), and reduced Duke (male infant head model) are shown, and the |B₁⁺| distribution is normalized to have an average of 1 μT. When the metasurface is applied, reduction rates of a CV of the |B₁⁺| distribution inside the ROI of the induced Duke, MIDA, and reduced Duke are 40% (reduced from 0.27 to 0.15), 46% (reduced from 0.37 to 0.20), and 44% (reduced from 0.31 to 0.17), respectively.

In FIG. 9B, a probability density function of a specific absorption rate (SAR) inside each head (including a region outside an ROI) when a metasurface optimized for Duke is not present and is present in a 7T magnetic resonance operating environment is shown. When the metasurface is applied, reduction rates of a peak SAR inside heads of induced Duke, MIDA, and reduced Duke are 5% (reduced from 3.89 W/kg to 3.69 W/kg), 9% (reduced from 4.66 W/kg to 4.25 W/kg), and 13% (reduced from 2.85 W/kg to 2.47 W/kg), respectively. In addition, reduction rates of an average SAR inside the heads are 6% (from 1.40 W/kg to 1.31 W/kg), 21% (from 1.36 W/kg to 1.07 W/kg), and 23% (from 1.20 W/kg to 0.93 W/kg), respectively. In FIG. 9, a normalized CV and a peak SAR are shown at a right side of each section.

FIG. 10 is a first flowchart illustrating a metasurface design method according to an embodiment of the present invention.

Hereinafter, a process of designing a metasurface will be described with reference to FIG. 10. Some operations described below may be performed in an order different from an order to be described below or may be omitted.

First, a processor 140 may acquire design information about an artificial magnetic field scatterer (S1001). The design information about the artificial magnetic field scatterer may include information about a structure of the artificial magnetic field scatterer.

Next, the processor 140 may acquire a plurality of high-frequency magnetic field modes that are distinguished from each other (S1003). While moving the artificial magnetic field scatterer model a preset distance (for example, 3 cm) in a preset direction (for example, a vertical direction), the processor 140 may repeatedly perform a process of modeling the artificial magnetic field scatterer based on the design information about the artificial magnetic field scatterer to generate an artificial magnetic field scatterer model, arranging the generated artificial magnetic field scatterer model in a virtual three-dimensional space, and then acquiring information about a distribution of a high-frequency magnetic field induced inside a subject, thereby acquiring the plurality of high-frequency magnetic field modes.

Next, the processor 140 may detect an optimal combination of the high-frequency magnetic field modes (S1005). A process for detecting the optimal combination of the high-frequency magnetic field modes will be described in detail below.

Next, the processor 140 may design an optimal metasurface based on the optimal combination (S1007). The processor 140 may determine a metasurface with a structure of a combination of magnetic field scatterers that induce an optimal combination of high-frequency magnetic field modes as an optimal metasurface (a metasurface with an optimal structure). That is, a metasurface designed according to the present embodiment may be a combination of magnetic field scatterers that induce an optimal combination of high-frequency magnetic field modes.

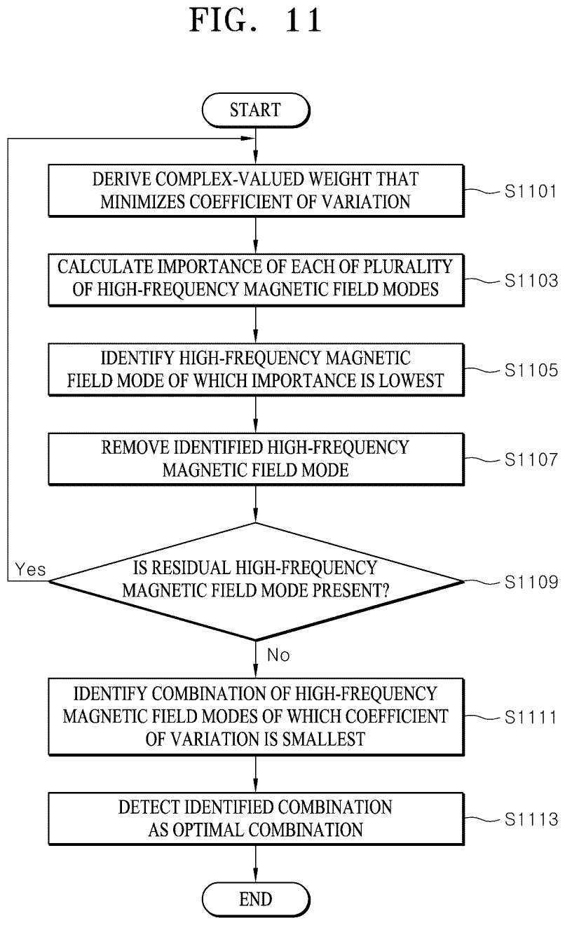

FIG. 11 is a second flowchart illustrating a metasurface design method according to an embodiment of the present invention.

Hereinafter, a process of detecting an optimal combination of high-frequency magnetic field modes will be described with reference to FIG. 11. Some operations described below may be performed in a different order than described below or may be omitted.

First, the processor 140 may derive a complex-valued weight that minimizes a CV (S1101). The homogenizing of a high-frequency magnetic field may be defined by a linear combination of complex-valued weights for each of a plurality of high-frequency magnetic field modes, and the complex-valued weights for each of the plurality of high-frequency magnetic field modes may be determined through a cost function for minimizing a CV for a distribution of an absolute value |B₁⁺| of the high-frequency magnetic field in an ROI for homogenizing. The processor 140 may adjust the complex-valued weight in a direction in which the CV is minimized. The processor 140 may derive a complex-valued weight that minimizes the CV based on a high-frequency magnetic field mode that is currently present.

Next, the processor 140 may calculate the importance of each of the plurality of high-frequency magnetic field modes (S1103). The importance of the high-frequency magnetic field mode may be determined according to an absolute value |Cₙ| of the complex-valued weight.

Next, the processor 140 may identify a high-frequency magnetic field mode of which the importance is the lowest (S1105) and may remove the identified high-frequency magnetic field mode (S1107).

Next, the processor 140 may determine whether a residual high-frequency magnetic field mode is present (S1109).

When the residual high-frequency magnetic field mode is present, the processor 140 may return to operation S1101. On the other hand, when the residual high-frequency magnetic field mode is not present, the processor 140 may identify a combination of high-frequency magnetic field modes of which the CV is the smallest CV (S1111).

Next, the processor 140 may detect the identified combination as an optimal combination of the high-frequency magnetic field modes (S1113).

In this way, according to the present invention, it is possible to design an optimal metasurface for achieving wide area homogenization of a high-frequency magnetic field transmitted to a brain of a subject.

In addition, according to the present invention, a portion of a metasurface of which the importance is low may be removed through pruning to simplify a structure of the metasurface, and stability may be imparted to B₁⁺ uniformity to allow the metasurface to be highly compatible with various subjects.

In addition, according to the present invention, by theoretically proving the effective material properties of an artificial magnetic field scatterer, it is possible to provide a high degree of freedom and flexibility in a design of a metasurface.

However, the effects that may be achieved through the present invention are not limited to the above-described effects, and other technical effects that are not described herein will be clearly understood by those skilled in the art from the following descriptions of the present invention.

As used herein, the terms “unit” and “module” may include a unit implemented in hardware, software, or firmware, and may interchangeably be used with other terms, for example, “logic,” “logic block,” “part,” or “circuitry.” “Part” and “module” may refer to components that are integrally formed or a minimum unit of the components that perform one or more functions or a part thereof. For example, according to one embodiment, a “unit” or “module” may be implemented in the form of an application-specific integrated circuit (ASIC).

Implementations described herein may be implemented in, for example, a method or process, an apparatus, a software program, a data stream, or a signal. Even when discussed only in the context of a single form of implementation (e.g., discussed only as a method), implementations of the discussed features may also be implemented in other forms (for example, an apparatus or a program). The apparatus may be implemented in suitable hardware, software, firmware, and the like. A method may be implemented in an apparatus such as a processor, which is generally a computer, a microprocessor, an integrated circuit, a processing device including a programmable logic device, or the like. Processors also include communication devices such as a computer, a cell phone, a portable/personal digital assistant (PDA), and other devices that facilitate communication of information between end-users.

Although the present invention has been described with limited embodiments and drawings, the present invention is not limited to thereto, and instead, it would be appreciated by those skilled in the art that various modifications and changes may be made to these embodiments without departing from the principles and spirit of the present invention, the scope of which is defined by the claims and their equivalents.

What is claimed is:

1. A metasurface design apparatus for designing a metasurface that is applied to a seven-tesla (7T) brain magnetic resonance imaging method, the metasurface design apparatus comprising:
a memory in which at least one instruction is stored; and
a processor configured to execute the at least one instruction stored in the memory, wherein the processor acquires design information about an artificial magnetic field scatterer, acquires a plurality of high-frequency magnetic field (B₁⁺) modes distinguished from each other using the acquired design information, detects an optimal combination of the high-frequency magnetic field modes for homogenizing a high-frequency magnetic field, and designs an optimal metasurface based on the detected optimal combination.

2. The metasurface design apparatus of claim 1, wherein the artificial magnetic field scatterer has an elliptical cylindrical structure including copper wires and parallel plate capacitors which are regularly arranged.

3. The metasurface design apparatus of claim 2, wherein effective material properties of the artificial magnetic field scatterer and magnetic field scattering by the artificial magnetic field scatterer are determined by a capacitance value of the parallel plate capacitor.

4. The metasurface design apparatus of claim 1, wherein, in the high-frequency magnetic field mode, a distribution of a high-frequency magnetic field induced inside a subject is exhibited by a radio frequency (RF) coil and the artificial magnetic field scatterer.

5. The metasurface design apparatus of claim 1, wherein a region of interest for the homogenizing is an entire brain region.

6. The metasurface design apparatus of claim 1, wherein the processor acquires the plurality of high-frequency magnetic field modes by repeatedly performing a process of modeling the artificial magnetic field scatterer based on the design information about the artificial magnetic field scatterer to generate an artificial magnetic field scatterer model, arranging the generated artificial magnetic field scatterer model in a virtual three-dimensional space, and then acquiring information about a distribution of a high-frequency magnetic field induced inside a subject while moving the artificial magnetic field scatterer model a preset distance in a preset direction.

7. The metasurface design apparatus of claim 1, wherein the homogenizing of the high-frequency magnetic field is defined by a linear combination of complex-valued weights for each of the plurality of high-frequency magnetic field modes.

8. The metasurface design apparatus of claim 7, wherein the complex-valued weights are determined through a cost function for minimizing a coefficient of variation for a distribution of an absolute value of the high-frequency magnetic field in a region of interest for the homogenizing, and the processor adjusts the complex-valued weights in a direction in which the coefficient of variation is minimized using a gradient descent method.

9. The metasurface design apparatus of claim 8, wherein, the processor repeatedly performs a process of deriving a complex-valued weight that minimizes the coefficient of variation, calculating importance of each of the plurality of high-frequency magnetic field modes, identifying a high-frequency magnetic field mode of which the importance is lowest, and removing the identified high-frequency magnetic field mode until the high-frequency magnetic field mode is no longer present, and then the processor identifies a combination of high-frequency magnetic field modes of which the coefficient of variation is smallest and detects the identified combination as the optimal combination.

10. The metasurface design apparatus of claim 9, wherein the importance is determined according to an absolute value of the complex-valued weight.

11. The metasurface design apparatus of claim 9, wherein, when there are a plurality of combinations of high-frequency magnetic field modes of which the coefficient of variation is smallest, a combination in which a number of included high-frequency magnetic field modes is smallest is detected as the optimal combination.

12. The metasurface design apparatus of claim 1, wherein the processor determines a metasurface with a structure of a combination of magnetic field scatterers that induce the optimal combination of the high-frequency magnetic field modes as the optimal metasurface.

13. A metasurface design method of designing a metasurface that is applied to a seven-tesla (7T) brain magnetic resonance imaging method, the metasurface design method comprising:
acquiring, by a processor, design information about an artificial magnetic field scatterer;
acquiring, by the processor, a plurality of high-frequency magnetic field (B₁⁺) modes distinguished from each other using the artificial magnetic field scatterer;
detecting, by the processor, an optimal combination of the high-frequency magnetic field modes for homogenizing a high-frequency magnetic field; and
designing, by the processor, an optimal metasurface based on the detected optimal combination.

14. The metasurface design method of claim 13, wherein, in the high-frequency magnetic field mode, a distribution of a high-frequency magnetic field induced inside a subject is exhibited by a radio frequency (RF) coil and the artificial magnetic field scatterer.

15. The metasurface design method of claim 13, wherein, in the acquiring of the plurality of high-frequency magnetic field modes, the processor acquires the plurality of high-frequency magnetic field modes by repeatedly performing a process of modeling the artificial magnetic field scatterer based on the design information about the artificial magnetic field scatterer to generate an artificial magnetic field scatterer model, arranging the generated artificial magnetic field scatterer model in a virtual three-dimensional space, and then acquiring information about a distribution of a high-frequency magnetic field induced inside a subject while moving the artificial magnetic field scatterer model a preset distance in a preset direction.

16. The metasurface design method of claim 13, wherein the homogenizing of the high-frequency magnetic field is defined by a linear combination of complex-valued weights for each of the plurality of high-frequency magnetic field modes.

17. The metasurface design method of claim 16, wherein the complex-valued weights are determined through a cost function for minimizing a coefficient of variation for a distribution of an absolute value of the high-frequency magnetic field in a region of interest for the homogenizing, and a gradient descent method is used to adjust the complex-valued weights in a direction in which the coefficient of variation is minimized.

18. The metasurface design method of claim 17, wherein, in the detecting of the optimal combination, the processor repeatedly performs a process of deriving a complex-valued weight that minimizes the coefficient of variation, calculating importance of each of the plurality of high-frequency magnetic field modes, identifying a high-frequency magnetic field mode of which the importance is lowest, and removing the identified high-frequency magnetic field mode until the high-frequency magnetic field mode is no longer present, and then the processor identifies a combination of high-frequency magnetic field modes of which the coefficient of variation is smallest and detects the identified combination as the optimal combination.

19. The metasurface design method of claim 18, wherein the importance is determined according to an absolute value of the complex-valued weight.

20. The metasurface design method of claim 13, wherein, in the designing of the optimal metasurface, the processor determines a metasurface with a structure of a combination of magnetic field scatterers that induce the optimal combination of the high-frequency magnetic field modes as the optimal metasurface.
